(19) United States
(12) Patent Application Publication
(10) Pub. No.: US 2018/0330043 A1
(43) Pub. Date: Nov. 15, 2018

(54) METHODS FOR THE HARVESTING OF CELL CULTURES

(71) Applicant: MILLENNIUM PHARMACEUTICALS, INC., Cambridge, MA (US)

(72) Inventors: Sheldon F. Oppenheim, Wellesley Hills, MA (US); Kevin C. Carrigan, Cambridge, MA (US)

(73) Assignee: MILLENNIUM PHARMACEUTICALS, INC., Cambridge, MA (US)

(21) Appl. No.: 15/997,158

(22) Filed: Jun. 4, 2018

Related U.S. Application Data
(63) Continuation of application No. 14/346,225, filed on Jul. 2, 2014.
(60) Provisional application No. 61/674,940, filed on Jul. 24, 2012.
(60) Provisional application No. 61/537,269, filed on Sep. 21, 2011.

(51) Int. Cl. G06F 19/10; C12N 5/00; G06F 17/18

(52) U.S. Cl. CPC G06F 19/10; G06F 17/18; C12N 2525/00; C12N 5/0081

(57) ABSTRACT

The present invention provides methods for optimization of the harvest process by clarification of cell samples using centrifugation and depth filtration. The present invention provides methods for the determination of the optimal ratio of Q/Σ for the centrifugation step of a harvest process of a cell culture. The present invention provides methods for the determination of the number of particles and the size of the particles in the centrate of a centrifugation step of a harvest process of a cell culture by the use of imaging technology. The present invention provides methods for the scaling of the harvesting process from lab-bench scale to industrial scale.

### CROSS-REFERENCE TO RELATED APPLICATIONS

This application is a continuation of application Ser. No. 14/346,225, filed Jul. 2, 2014, which is a national stage application under 35 U.S.C. § 371 of International Application No. PCT/US2012/055035, filed Sep. 13, 2012, which designated the U.S. and claims the benefit of priority of U.S. Provisional Application No. 61/674,940, filed Jul. 24, 2012, and U.S. Provisional Application No. 61/537,269, filed Sep. 21, 2011, all of which are incorporated herein by reference.

### FIELD OF THE INVENTION

The invention relates generally to methods for solid-liquid separation, for example, separation of cells and cellular debris in a biological sample to thereby prepare a clarified, cell-free sample. The methods described herein are useful during the harvesting process of proteins or antibodies generated by cells in culture.

### BACKGROUND OF THE INVENTION

The harvest unit operation most often used for the primary recovery of cell-based biopharmaceuticals typically consists of continuous centrifugation followed by a single or multistep depth filtration. The primary function of the harvest process is to separate process solids (cells and cell debris) from the process (bioreactor) supernatant, although the depth filter portion of the harvest process can also impact host cell protein and DNA levels [Yigzaw Y., Piper R., Tran M., and Shukla A. A., (2006) Exploitation of the Adsorptive Properties of Depth Filters for Host Cell Protein Removal During Monoclonal Antibody Purification. Biotechnol. Prog. 22: 288-296]. It has also been reported that the depth filtration has the capability to provide some clearance of adventitious viruses [Tipton B., Boose J. A., Larsen W., Beck J., and O'Brien T., (2002) Retrovirus and Parvovirus Clearance from and Affinity Column Product Using Adsorptive Depth Filtration. BioPharm. September; 43-50].

A typical harvest process is composed of disc-stack centrifugation followed by depth filtration using filters based on diatomaceous earth [See for example: Russell, E., (2003) Evaluation of Disc Stack Centrifugation for Clarification of Mammalian Cell Culture. Master's Thesis; Tufts University. 3-4; David, Y., Blanck, R., Lambalot, C., and Brunkow R., (2003). The Clarification of Bioreactor Cell Cultures for Biopharmaceuticals. Pharma. Tech. March; pp 62-76; Purav, D., Dizon-Maspat, J., and Cano, T., (2009). Evaluation and Implementation of a Single-Stage Multimedia Harvest Depth Filter for a Large-Scale Antibody Process. BioPharm. Int. June 8-17].

One of the common problems encountered is the scaling up of pilot centrifuge settings to production/industrial scale. It might seem that scaling based on relative centrifugal force would be appropriate, but this fails to account for geometric differences between centrifuges. Thus it is more appropriate to scale based on the concept of equivalent settling area [Boychyn, M., Yim, S. S. S., Bulmer M., More .J, Bracewell D. G., Hoare M., (2004) Performance prediction of industrial centrifuges using scale-down models. Bioprocess and Biosystems Engineering 26:385-391]. Based on this concept, the centrifugation process should be scaled using a combination of the flow rate (Q) and the angular velocity as espressed by Equation 1 below [Doran, P. M, (1997). Bioprocess Engineering Principles. San Diego: Academic Press. 228-229; Ambler, C. M., (1952). The Evaluation of Centrifugation Performance. Chem. Eng. Prog. 48: 150-158]:

$$\frac{{Q}_{old}}{{Σ}_{old}} = \frac{{Q}_{new}}{{Σ}_{new}} \\ where \\ Σ = \frac{2 {πω}^{2} ( N \text{-} 1 )}{3 g tan θ} ( {r}_{2}^{3} - {r}_{1}^{3} ) \qquad ( Equaton 1 )$$

Q=flow rate
ω=angular velocity
N=number of discs
g=gravitational acceleration
r₁=inner radius of a disc
r₂=outer radius of a disc

The centrifuge typically removes larger particles or solids which are accumulated along the walls of the bowl and are periodically discharged. The centrifugation step is typically followed by one or two stages of depth filtration. The depth filter removes particles by several modes including electrostatic effects, mechanical retention, and particle size exclusion [Purav, D., Dizon-Maspat, J., and Cano, T., (2009). Evaluation and Implementation of a Single-Stage Multimedia Harvest Depth Filter for a Large-Scale Antibody Process. BioPharm. Int. June 8-17; Doran, P. M, (1997). Bioprocess Engineering Principles. San Diego: Academic Press. 386]. Commercially available filters provide not only particle removal but may also remove initial host cell protein, DNA and viral reduction.

The typical tool for analyzing the success of the harvest process is a turbidity measurement. Turbidity was defined in the 1999 Environmental Protection Agency (EPA) guidance manual as: Turbidity is a principal physical characteristic of water and is an expression of the optical property that causes light to be scattered and absorbed by particles and molecules rather than transmitted in straight lines through a water sample (EPA Guidance Manual: Turbidity Provisions April 1999. Chapter 7-1). The later 2004 EPA Guidance manual simplified the definition to: Turbidity is the measure of how clear a liquid is and how much light is scattered by the sample (LT1ESWTR EPA Guidance Manual: Turbidity Provisions August 2004. Chapter 7-1). If the term water or liquid is replaced with process or bioreactor supernatant this definition can be applied to the harvest process and turbidity becomes in essence a measure of the cloudiness of the solution. Typically, this cloudiness is measured against Formazin standards and the result is often expressed in Nephelometric Turbidity Units (NTU). The actual device that measures the turbidity of a solution projects light (the type of light is method dependent) through a sample and a sensor detects the intensity of the light passed through the sample as well as scattered at 90° to the light source [Sader, M. J. (1998). TURBIDITY SCIENCE: Technical Information Series—Booklet No. 11. Hach Company, Loveland Colo. 7-8].

Depending on the method employed, light scatter (at other angles) may also be measured and factored into the final output. The instruments and standards are inexpensive and readily available from several vendors. However, results from different style units and different Formazin standards are often not comparable. Thus, applying turbidity to the harvest process can reveal that each sub-step of this unit operation is reducing or not reducing cloudiness (particles). Thus, monitoring the turbidity during the process can only indicate that there is a change, but not the reason for the change.

Thus, there remains a need for additional methods to understand and troubleshoot and optimize the entire harvest process.

### SUMMARY OF THE INVENTION

The present invention provides methods for optimization of the harvest process by clarification of cell samples using centrifugation and depth filtration. For example, these methods are useful for clarification of bacterial, insect and mammalian cell cultures. The methods described herein use imaging technology to determine parameters of the particles present in the centrate or in the filtrate of depth filters. Such parameters are useful to minimize clogging of the depth filter, thereby improving efficiency and reducing the cost of the harvest process.

### BRIEF DESCRIPTION OF THE DRAWINGS

FIG. 1 shows a typical image of unprocessed bioreactor supernatant.

FIG. 2 shows the reduction of the average number of particles per analyzed frame in Example 1.

FIG. 3 shows the average particle diameter of centrate in Example 1.

FIG. 4 shows the distribution of specified particle size ranges throughout the harvest process in Example 1.

FIG. 5 shows the distribution of particle diameter ranges from the centrifugation conditions tested in Example 2.

FIG. 6 shows a sizing scheme using particle counting technology.

### DETAILED DESCRIPTION OF THE INVENTION

In one aspect, the present invention provides methods for the clarification of cell samples or cell cultures using centrifugation and depth filtration.

In another aspect, the present invention provides methods for the determination of the optimal ratio of Q/Σ for the centrifugation step of a harvest process of a cell culture.

In another aspect, the present invention provides methods for the determination of the number of particles and the size of the particles in the centrate of a centrifugation step and/or the filtrate of the depth filtration step of a harvest process of a cell culture by the use of imaging technology.

In another aspect, the present invention provides methods for the scaling of the harvesting process from lab-bench scale to industrial scale.

In another aspect, the present invention provides methods for the continuous monitoring of the degradation of depth filters during a harvesting process.

As used herein, the term cell culture can be a cell suspension or a cell slurry, and can include cell culture of between 1 liter and 20,000 liters.

As used herein, the centrifugation step may be performed using any centrifuge (lab-scale or industrial scale). In some embodiments, the centrifuge employed is a disc-stack centrifuge. In some embodiments, the centrifuge employed is a continuously operated disc-stack centrifuge. In some embodiments, the centrifuge employed is an automated discharge disc-stack centrifuge. In some embodiments, the centrifuge employed is a non-automated disc-stack centrifuge. In some embodiments, the centrifuge is a CTC-1 centrifuge, a Whisperfuge centrifuge, a SA-1 centrifuge, a CSC-6 centrifuge, a SC-6 centrifuge or a CSC-20 centrifuge. In some embodiments, the depth filter is a 3M dual layer ZetaPlus series 120ZA05A or 90ZA05A depth filter, a Millipore A1HC or B1HC depth filter, or a Pall PDE0, PPD1 or PDE2 depth filter.

Cell cultures may be bacterial cell cultures, insect cell cultures, yeast cell cultures or mammalian cell cultures. In some embodiments, the cell culture is a bacterial cell culture. In some embodiments, the cell culture is an insect cell culture. In some embodiments, the cell culture is a yeast cell culture. In some embodiments, the cell culture is a mammalian cell culture. In some embodiments, the mammalian cells employed in the cell culture are CHO cells, NS0 cells, Sp20 cells or PerC6 cells. In some embodiments, the mammalian cells employed in the cell culture are CHO cells. In some embodiments, the mammalian cells employed in the cell culture are NS0 cells. In some embodiments, the mammalian cells employed in the cell culture are PerC6 cells. In some embodiments, the mammalian cells employed in the cell culture are Sp20 cells.

The information that is not available from turbidity measurements is the number and size of particles and statistical distribution of the particles remaining in or removed from the process supernatant during each step of the harvest. It is desirable to reduce the number of particles remaining in the process supernatant as well as to minimize the size of the particles remaining in the supernatant in order to minimize the likelihood of clogging the depth filter during the subsequent depth filtration step.

In some embodiments, the method comprises the use of imaging technology to analyze the centrate of a centrifugation step of a harvest process of a cell culture to determine centrifugation conditions that achieve the lowest number of particles in the centrate and the smallest size of particles in the centrate. In some embodiments, the method comprises the use of imaging technology to analyze the centrate of a centrifugation step of a harvest process of a cell culture to determine centrifugation conditions that achieve the lowest number of particles in the centrate. In some embodiments, the method comprises the use of imaging technology to analyze the centrate of a centrifugation step of a harvest process of a cell culture to determine centrifugation conditions that achieve the desired size and number of particles in the centrate.

Design of experiment (DOE) is a statistical method that explores the interaction of factors that control a process or system. DOE shows how interconnected factors respond over a wide range of values without having to test all the values directly. DOE takes response data from a process or system and fits the data to mathematical equations and then uses the equations as models to predict what will happen for any given combination of values. DOE software is available from multiple vendors including JMP (SAS, Cary, N.C., US); S-matrix (Eureka Calif., US) Stat Ease (Minneapolis, Minn., US) and Essential Regression and Experimental Design (Dave Steppan, Joachim Werner and Bob Yeater. www.oocities.org/siliconvalley/network/1032/).

The flow rate (Q) of a centrifugation or depth filtration procedure is measured by the volumetric flow. The volumetric flow rate is the volume of solution collected in a given amount of time and is represented in the units of volume per unit time. In one embodiment, the flow rate of the centrifugation step is the same as the flow rate of the depth filtration step. In another embodiment, the flow rate of the centrifugation step is different from the flow rate of the depth filtration step. The flux is the volumetric flowrate per unit cross-sectional area of a given depth filter.

In some embodiments, the method for determining the optimal Q/Σ ratio for a centrifugation step of a harvest process of a cell culture comprises:
(i) performing a DOE experiment on a centrifugation step employing x input parameters;
(ii) obtaining samples from the centrate of the centrifugation step for each of the experiments;
(iii) analyzing the samples for y output parameters;
(iv) employing a statistical analysis to generate the optimal Q/Σ ratio (i.e., the optimal ratio for the best performance of the unit operation) using equation (1).

In some embodiments, the method for determining the optimal Q_{new}/Σ_{new} ratio for a centrifugation step of a harvest process of a cell culture comprises:
(i) determining the value of Q_{old} using a lab-scale depth filtration device;
(ii) performing a DOE experiment on a lab-scale centrifugation step employing x input parameters;
(iii) obtaining samples from the centrate of the centrifugation step for each of the experiments;
(iv) analyzing the samples for y output parameters;
(v) employing a statistical analysis to generate the optimal Q_{old}/Σ_{old} ratio;
(vi) determining the value of Q_{new} using an industrial scale depth filtration device; and
(vii) determining the value of Σ_{new} using equation (1).

In some embodiments, the optimal Q/Σ ratio will be determined for the same centrifugation apparatus that was used in the DOE experiment. In some embodiments, the optimal Q/Σ ratio will be determined for a different centrifugation apparatus. In some embodiments, the optimal Q/Σ ratio will be used to scale-up the centrifugation process from a lab-bench scale to an industrial scale.

Imaging systems that utilize charge-coupled device (CCD) sensors and complementary metal oxide semi-conductor (CMOS) active pixel sensors to determine particle size distribution and number of particles are available from several vendors including J M Canty, Inc. (Buffalo, N.Y., US), Cilas (www.cilas.com), Prozesstechnik (Oberkotzau, Germany), Protein Simple (Santa Clara, Calif., 95051 USA), and Malvern Instruments (Worcestershire, UK). Many of these systems are available with flow cells. For example, a micro-CCD sensor unit (MicroFlow™) (J M Canty, Inc.).

Some units use laser and optical analysis whereas others just use optical analysis. The optical unit acts as a video microscope and can operate in several different microscopy modes (back-lit, dark-field, and cross polarization). In some embodiments, a laser and optical analysis unit is employed. In some embodiments, an optical analysis is employed. In some embodiments, a flow-through optical unit is employed.

The image analysis software receives and analyzes the data from the optical unit. In order for the software to provide quantitative information, a reticle (a grid of defined spacing, typically etched on a piece of glass) is needed to calibrate the distance measurement. The CCD sensor unit measures the light intensity per pixel and the software analyzes a frame to calculate output parameters including: particle diameter distribution, the number of particles per frame (which can be converted into particles/mL), a mean diameter, particle perimeter, size distribution, aspect ratio and the particle roundness.

In some embodiments, the optical unit and image analysis software analyzes samples taken from the centrate during the operation of the centrifugation. In some embodiments, the optical unit and image analysis software are set up in-line as part of the process. In some embodiments, the set-up is equipped to send out an alarm should a defined parameter be out of a pre-set specification. In some embodiments, the set-up is equipped to adjust the flow rate should a defined parameter be out of pre-set specifications.

In some embodiments, the number of output parameters, y, is 1-10. In some embodiments, the number of output parameters, y, is 1-5. In some embodiments, the number of output parameters, y, is 5. In some embodiments, the number of output parameters, y, is 4. In some embodiments, the number of output parameters, y, is 3. In some embodiments, the number of output parameters, y, is 2. In some embodiments, the number of output parameters, y, is 1.

In some embodiments, the output parameters determined by the image analysis software, y, comprise one or more of the output parameters selected from the group consisting of:
(i) the number of particles per frame;
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

In some embodiments, the output parameters determined by the image analysis software, y, comprise:
(i) the number of particles per frame;
(ii) the average chord length of the particles;
(iii) the minimum centroid diameter of the particles;
(iv) the equivalent circular diameter of the particles; and
(v) the statistical distribution of particle size versus the number of particles at said size.

In some embodiments, the output parameters determined by the image analysis software, y, comprise:
(i) the number of particles per frame; and optionally one or more of
(ii) the average chord length of the particles;
(iii) the minimum centroid diameter of the particles;
(iv) the equivalent circular diameter of the particles; and
(v) the statistical distribution of particle size versus the number of particles at said size.

As used herein, the output parameters determined by the image analysis software (i.e., CantyVisionClient™), have the meanings as defined by the manufacturer (see CantyVisionClient™ Manual, J M Canty Inc., Buffalo, N.Y., USA).

In some embodiments, the number of input parameters, x, is 1-10. In some embodiments, the number of input parameters, x, is 1-5. In some embodiments, the number of input parameters, x, is 5. In some embodiments, the number of input parameters, x, is 4. In some embodiments, the number of input parameters, x, is 3. In some embodiments, the number of input parameters, x, is 2. In some embodiments, the number of input parameters, x, is 1.

In some embodiments, the input parameters used in the DOE analysis, x, comprise one or more of the input parameters selected from the group consisting of:
(i) Q, flow rate
(ii) g, relative centrifugal force (RCF) of the centrifuge unit employed;
(iii) cell viability of the cell culture;
(iv) total cell count; and
(v) percent solids by volume.

In some embodiments, the input parameters used in the DOE analysis, x, comprise:
(i) Q, flow rate
(ii) g, relative centrifugal force (RCF) of the centrifuge unit employed; and
(iii) cell viability of the cell culture.

In some embodiments, the input parameters used in the DOE analysis, x, comprise:
(i) Q, flow rate;
(ii) g, relative centrifugal force of the centrifuge unit employed; and optionally one or more of:
(iii) cell viability of the cell culture;
(iv) total cell count; and
(v) percent solids by volume.

In some embodiments, the output parameters used in the DOE analysis, y, comprise one or more of the output parameters selected from the group consisting of:
(i) the number of particles per frame;
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

In some embodiments, the output parameters used in the DOE analysis, y, comprise:
(i) the number of particles per frame;
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

In some embodiments, the output parameters used in the DOE analysis, y, comprise:
(i) the number of particles per frame; and optionally one or more of:
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

In some embodiments, the output parameters used in the DOE analysis, y, comprise:
(i) the number of particles per frame;
(ii) the average chord length of the particles;
(iii) the minimum centroid diameter of the particles;
(iv) the equivalent circular diameter of the particles; and optionally
(v) turbidity.

### EXAMPLES

### Example 1

One hundred litres of a mammalian (CHO) cell based harvest broth was processed through a disc-stack centrifuge into a multilayer, single-stage, depth filter. Centrifugation samples were taken at approximately 2 and 12 minutes into a 14 minute centrifugation cycle. Additionally, three samples were taken exiting the depth filter over a 3 hour time period. The individual samples were circulated through the CCD sensor unit for approximately five minutes in order to obtain 3000 frames of data. The CCD sensor unit used was in the backlight mode. Every tenth frame was analyzed using the image processing software. The system used was able to identify particles ranging from 0.4-480 μm.

Prior to the execution of the experiment, the appropriate zoom level, distance calibration, and image intensity were determined and set. The data were processed in a serial manner. The turbidity of each sample was measured using a laser based turbidimeter calibrated with Stablcal Formazin standards.

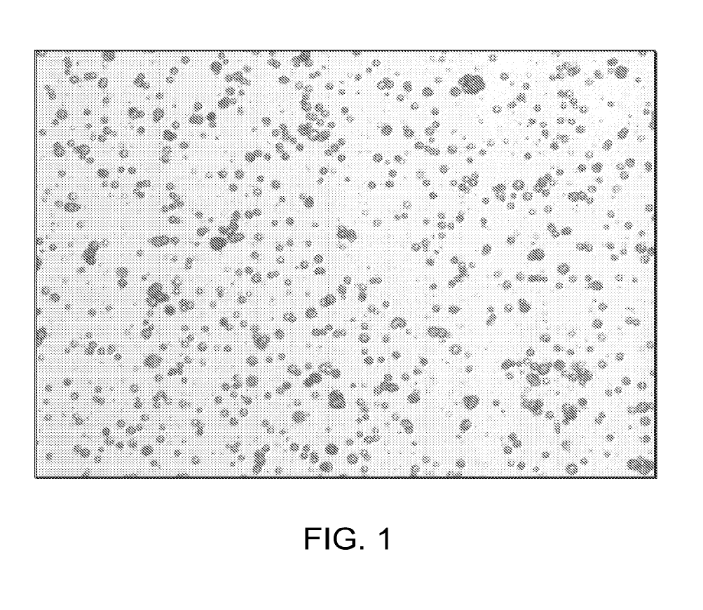

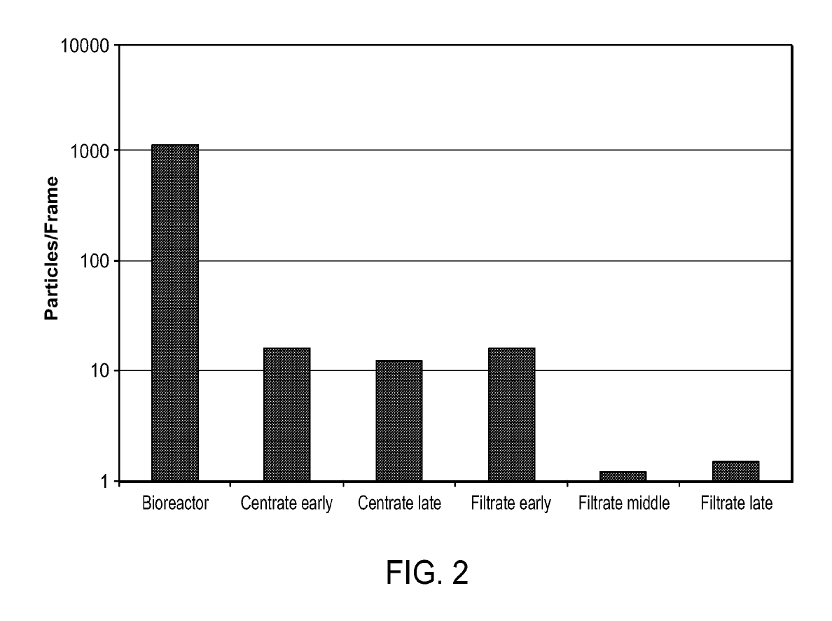

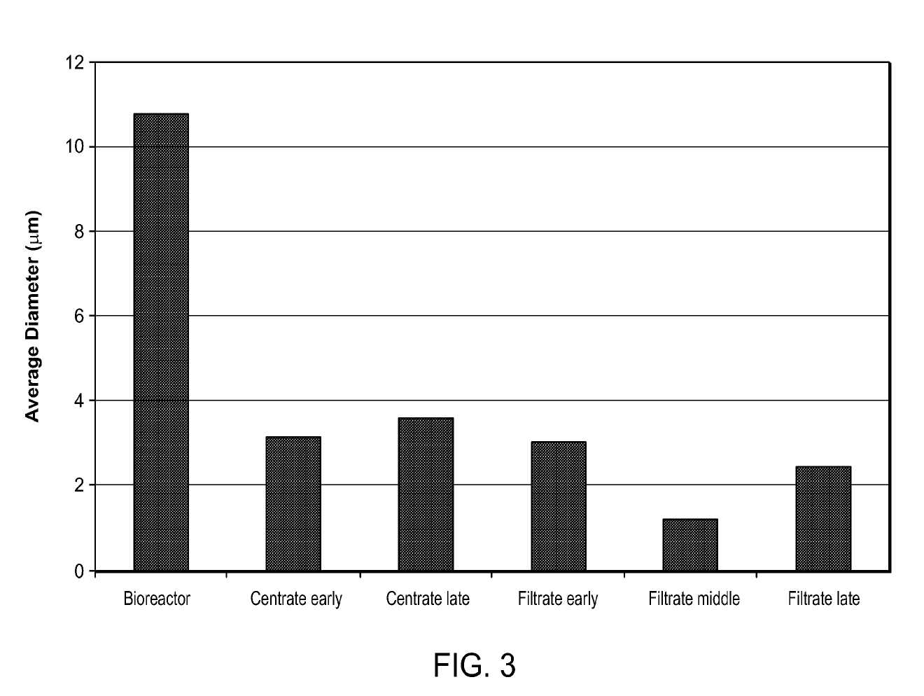

A typical image of unprocessed bioreactor supernatant is shown in FIG. 1 and shows a combination of single cells, aggregated cells and cellular debris (FIG. 1). As a result of the centrifugation step, the number of particles is reduced by ˜12-fold over the unprocessed material, as indicated by the reduction of the average number of particles per analyzed frame (FIG. 2). Interestingly, the number of particles increased initially again from the centrifugation step to the early stages of the depth filtration. Concomitantly with the decrease in the number of particles, the average particle diameter is decreased by a factor of ˜3.5 (FIG. 3). In contrast to the particle number, the average particle diameter does not increase from the centrifugation step to the early stages of the depth filtration. For comparison, a control sample of unprocessed cell culture fluid was also circulated through the CCD sensor unit and analyzed via the image processing system. In parallel to the image analyses, turbidity measurements were also taken and they were consistent with the particle counting data. An average value of 200 NTUs (unprocessed), 20 NTUs (centrate), and 2 NTUs (depth filtrate) were obtained for this harvest process.

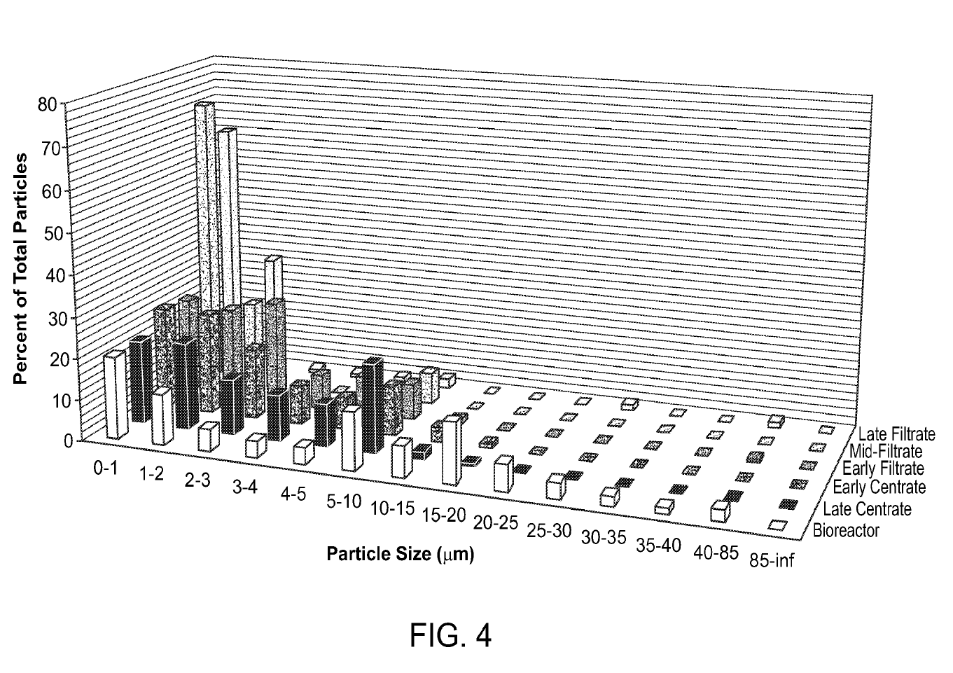

The average particle diameter data (FIG. 3) provides a good overview of the size of the remaining particles throughout each step of the process. But these data also allow a more detailed look at the distribution of specified particle size ranges throughout the harvest process (FIG. 4). Interestingly, the unprocessed bioreactor material shows a bimodal distribution of particle sizes. Particles smaller than 10 μm in diameter are usually associated with cell fragments whereas individual cells are expected in the range of 10-20 μm. From the data obtained it is apparent that, while the number of particles continues to decrease throughout the harvest process, those that remain in the solution are typically 5 μm in size or smaller (FIG. 4). A more detailed analysis of the particles smaller than 5 μm (at resolution of 1 μm) further showed a shift in the distribution to smaller particle sizes.

Based on these data, the centrifuge is able to remove the majority of particles that are 5 μm in size or larger. The depth filter used for the experiment did further decrease the overall number of particles in the process fluid and the size distribution of the particles became more skewed toward the smaller particle sizes.

### Example 2

The proof-of-concept study described in Example 1 above shows that analyzing the centrate of the centrifugation step using a CCD sensor unit is useful for developing a harvest process with an optimal Q/Σ ratio for the centrifugation step. This is the key parameter used for scaling of centrifugation operations on the concept of equivalent settling area.

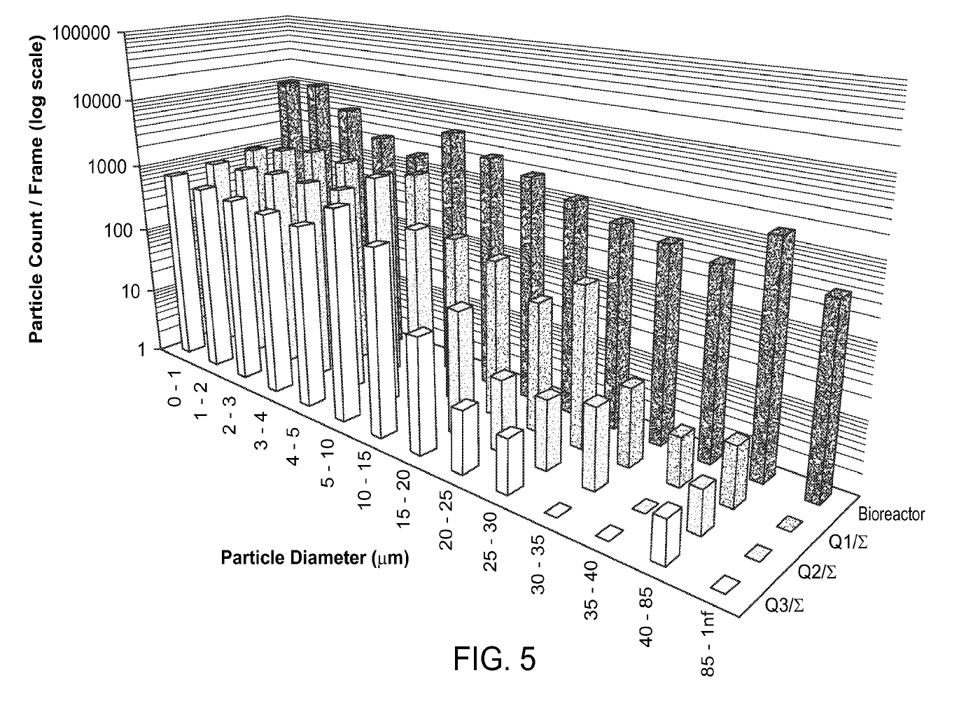

To further test the capability of CCD sensor units to optimize the centrifugation step of a harvest operation, the Q/Σ values for a harvest process on a lab-scale disc stack centrifuge were varied and the centrates were analyzed via particle counting and turbidity measurements. Three Q/Σ values were tested. The results show that Q/Σ at setting #3 had the lowest number of particles in the centrate (FIG. 5), which was consistent with the turbidity measurements indicating the lowest “cloudiness” for this sample (Table 1). These data demonstrate that we could detect changes in centrifugation performance by changing the input flow rate (Q). Consistent with the results obtained in Example 1 above, the majority of the remaining particles were smaller than 5 μm.

TABLE 1

| | | | | | Early | Late |
|---|---|---|---|---|---|---|
| | Bioreactor | Q₁/Σ | Q₂/Σ | Q₃/Σ | Filtrate | Filtrate |
| Turbidity | 1228 | 85.3 | 51.4 | 49.6 | 5.2 | 8.83 |
| (NTU) | | | | | | |

### Example 3

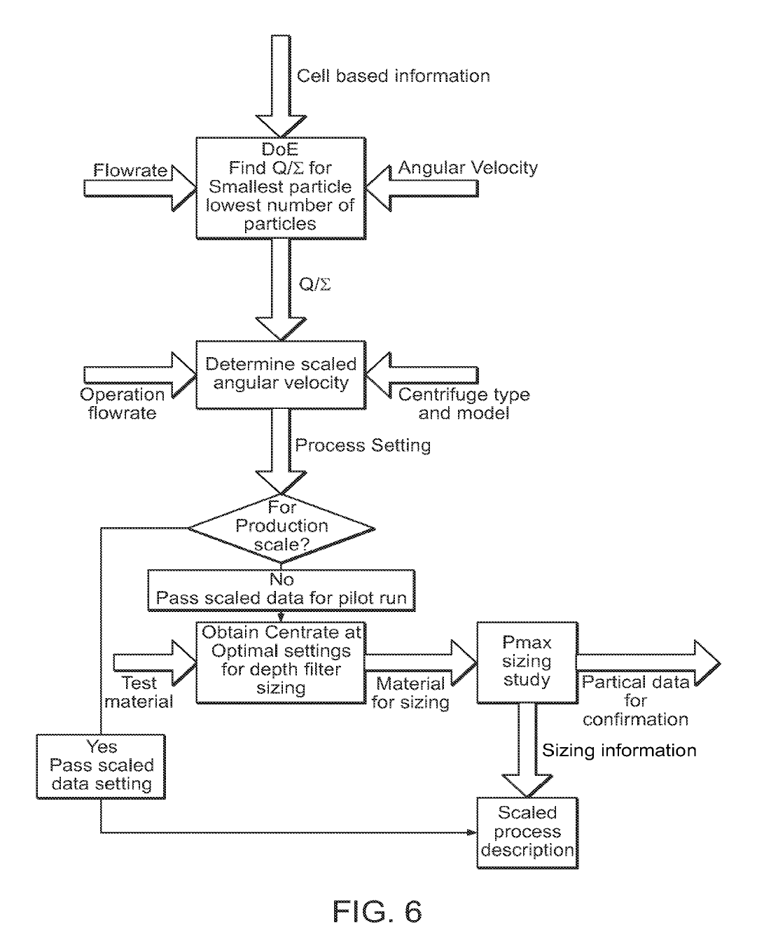

The following procedure provides an example of how one can determine the optimum relative centrifugal force (RCF) for the centrifugation of a scaled up harvest process using data obtained from a small scale experiment whereby a CCD sensor unit is used to analyze the output of the centrate (FIG. 6).

### General Procedure

(a) Harvesting Process

Prior to conducting the experiment, one needs to determine the DOE input parameters to be tested. The lab scale centrifuge is then set up in a manner in which samples may be collected. One then determines one or more of the following parameters for the material to be harvested: (i) cell viability, (ii) total cell count and/or (iii) percent solids by volume. Material is then placed in a collection container or the procedure is operated out of the bioreactor and kept at constant agitation to prevent settling of the cells. The centrifugation is then started at the first RCF condition. At the first Q setting, the pump is started and a stable back pressure is established. Samples are collected (˜50 mL), identified and kept for analysis. The Q and RCF settings are then changed to the next desired settings and samples are collected after establishing a stable back pressure. Repeat procedure as needed.

(b) Prior to Analysis

Prior to any sample analysis, one needs to calibrate the measurement of turbidity as well as the CCD sensor unit.

(c) Analytics

In order to analyze the samples obtained during the harvesting process, the turbidity of the samples (NTU) is measured as per manufacture's protocol. The NTU of each sample is recorded. Next, the samples are circulated through the CCD sensor unit at ˜75 mL/min. After approximately three minutes of circulation, obtain a stable image on the CCD sensor unit without touching the zoom and capture a representative image and set density. Using the CCD sensor unit, capture a movie of the sample (500-3000 frames total). Wash out the CCD sensor unit and then repeat with the next sample (ensure image has stabilized before starting to capture movies). Flush the CCD sensor unit after the last sample has been analyzed.

(d) Image Analytics

Once all of the movies have been captured, the captured movies are analyzed using a particle analysis software using a predetermined intensity in order to provide the output data of the experiment.

(e) Model Computation

The output data is compiled in a DOE software (e.g., JMP). The output data is then analyzed to determine suitable or preferred parameters for the small scale model, i.e., Q_{old}/Σ_{old}. The value of Q_{new} is then determined using data obtained from Pmax filter studies of the industrial scale depth filter. Pmax is the preferred test method when a constant flow test is performed and size exclusion is the primary method of particle removal. The capacity of the filter is determined by a pressure endpoint. The Pmax sizing method involves determining the filter resistance to flow as a function of throughput. Once Q_{new} has been determined, the RCF for the scaled up harvesting process can be determined via equation 1 recited above.

### Results

Using the general procedure described above, the optimal harvesting conditions for a scaled up harvesting process were determined using a small scale experiment using a CHO cell line culture and the parameters outlined in Table 2 below.

TABLE 2

| | | | | | | Average | minimum |
|---|---|---|---|---|---|---|---|
| | | | Average | | | equivalent | average |
| | | | Chord | | | circular | centroid |
| RCF | Q | Viability | Length | Particles | Turbidity | diameter | diameter |
| (g) | (mL/min) | (%) | (micron) | per frame | (NTU) | (micron) | (micron) |
| 8000 | 750 | 46.3 | 3.4 | 223 | 43 | 3.1 | 2.54 |
| 9000 | 1000 | 4 | 4.1 | 130 | 36 | 3.8 | 3.2 |
| 8000 | 1250 | 46.3 | 3.8 | 138 | 36.7 | 3.4 | 2.8 |
| 10000 | 750 | 4 | 4.3 | 104 | 32.9 | 3.9 | 3.4 |
| 10000 | 1250 | 4 | 4.2 | 152 | 43.5 | 3.9 | 3.3 |
| 8000 | 1000 | 46.3 | 3.6 | 195 | 33.3 | 3.2 | 2.6 |
| 9000 | 750 | 4 | 3.7 | 373 | 97.2 | 3.4 | 2.9 |

The output parameters were analyzed using the JMP software. Two small scale models were identified using the data of this small scale experiment (Table 3).

TABLE 3

| | Suggested settings | Suggested settings |
|---|---|---|
| | for Model 1 | for Model 2 |
| Q (mL/min) | 1000 | 1000 |
| RCF (g) | 9000 | 9000 |
| % Cell Viability | 24 | 25.1 |

Pmax filter studies were conducted on the industrial scale depth filter. These studies established that the Q_{new} value for the scaled up harvesting process is 13.3 L/min. Using the data from the two models above and Equation (1) outlined above, a value of 7078×g was determined for the scaled-up harvesting process.

All publications cited herein are hereby incorporated by reference. Unless defined otherwise, all technical and scientific terms used herein have the same meaning as commonly understood to one of ordinary skill in the art to which this invention pertains.

1-24. (canceled)

25. A method of harvesting a protein from a cell culture comprising:
(a) providing a portion of the cell culture;
(b) performing disk-stack centrifugation on the cell culture to obtain a centrate comprising the protein;
(c) visualizing the centrate using imaging technology to confirm at least a reduced number of particles in the centrate relative to the number of particles in the cell culture prior to centrifugation; and
(d) depth filtering the centrate to obtain a filtrate comprising the protein,
thereby harvesting the protein from the cell culture,
wherein the centrifugation step is performed at a flow rate/equivalent settling area (Q/Σ) ratio that separates cells and cellular debris from the centrate with a reduced number of particles, and wherein the Q/Σ ratio has been determined by:
(i) performing a design of experiment (DOE) statistical analysis on a centrifugation step employing x input parameters;
(ii) obtaining samples from the centrate of the centrifugation step for each DOE statistical analysis;
(iii) analyzing the samples for y output parameters;
(iv) employing a statistical analysis to generate the Q/Σ ratio;
wherein
x is 1-10; and
y is 1-10;
and wherein the imaging technology comprises a charge-coupled device (CCD)-sensor unit or a complementary metal oxide semi-conductor (CMOS)-sensor unit.

26. The method of claim 25, wherein the cell culture is a bacterial cell culture, an insect cell culture or a mammalian cell culture.

27. The method of claim 25, wherein the cell culture is a mammalian cell culture.

28. The method of claim 27, wherein the mammalian cell culture is comprised of CHO cells or NS0 cells.

29. The method of claim 25, wherein x is 1-5.

30. The method of claim 29, wherein the input parameters comprise:
(i) Q, flow rate;
(ii) g, relative centrifugal force (RCF) of the centrifuge unit employed; and
optionally one or more of:
(iii) cell viability of the cell culture;
(iv) total cell count; and
(v) percent solids by volume.

31. The method of claim 29, wherein the input parameters comprise:
(i) Q, flow rate
(ii) g, relative centrifugal force (RCF) of the centrifuge unit employed; and
(iii) cell viability of the cell culture.

32. The method of claim 25, wherein y is 1-5.

33. The method of claim 25, wherein the imaging technology comprises a CCD-sensor unit.

34. The method of claim 33, wherein the CCD-sensor unit is in-line and capable of analyzing samples during operation of the centrifugation step.

35. The method of claim 25, wherein the output parameters comprise:
(i) the number of particles per frame; and optionally one or more of:
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

36. The method of claim 35, wherein output parameters (i) and (ii)-(vi) if used are measured by a CCD-sensor unit.

37. A method of harvesting a protein from an industrial scale cell culture comprising:
(a) providing a portion of the cell culture;
(b) performing disk-stack centrifugation on the cell culture to obtain a centrate comprising the protein;
(c) visualizing the centrate using imaging technology to confirm at least a reduced number of particles in the centrate relative to the number of particles in the cell culture prior to centrifugation; and
(d) depth filtering the centrate to obtain a filtrate comprising the protein,
thereby harvesting the protein from the cell culture,
wherein the centrifugation step is performed at a Q_{new}/Σ_{new} ratio that separates cells and cellular debris from the centrate with a reduced number of particles, and
wherein the Q_{new}/Σ_{new} ratio has been determined by:
(i) determining the value of Q_{old} using a lab-scale depth filtration device;
(ii) performing a design of experiment (DOE) statistical analysis on a lab-scale centrifugation step employing x input parameters;
(iii) obtaining samples from the centrate of the centrifugation step for each DOE statistical analysis;
(iv) analyzing the samples for y output parameters;
(v) employing a statistical analysis to generate the Q_{old}/Σ_{old} ratio for the lab-scale centrifugation step;
(vi) determining the value of Q_{new} using an industrial scale depth filtration device; and
(vii) determining the value of Σ_{new} using equation (1);
wherein
x is 1-10; and
y is 1-10;
and wherein the imaging technology comprises a charge-coupled device (CCD)-sensor unit or a complementary metal oxide semi-conductor (CMOS)-sensor unit.

38. The method of claim 37, wherein the cell culture is a bacterial cell culture, an insect cell culture or a mammalian cell culture.

39. The method of claim 37, wherein the cell culture is a mammalian cell culture.

40. The method of claim 39, wherein the mammalian cell culture is comprised of CHO cells or NS0 cells.

41. The method of claim 37, wherein x is 1-5.

42. The method of claim 37, wherein the input parameters comprise:
(i) Q, flow rate;
(ii) g, relative centrifugal force (RCF) of the centrifuge unit employed; and
optionally one or more of:
(iii) cell viability of the cell culture;
(iv) total cell count; and
(v) percent solids by volume.

43. The method of claim 37, wherein the input parameters comprise:
(i) Q, flow rate;
(ii) g, relative centrifugal force (RCF) of the centrifuge unit employed; and
(iii) cell viability of the cell culture.

44. The method of claim 37, wherein y is 1-5.

45. The method of claim 37, wherein the imaging technology comprises a CCD-sensor unit.

46. The method of claim 45, wherein the CCD-sensor unit is in-line and capable of analyzing samples during operation of the centrifugation step.

47. The method of claim 37, wherein the output parameters comprise:
(i) the number of particles per frame; and optionally one or more of:
(ii) the average chord length of the particles;
(iii) the minimum centroid diameter of the particles;
(iv) the equivalent circular diameter of the particles;
(v) the statistical distribution of particle size versus the number of particles at said size; and
(vi) turbidity.

48. The method of claim 47, wherein output parameters (i) and (ii)-(iv) if used are measured by a CCD-sensor unit.
